(19) United States
(12) Patent Application Publication
(10) Pub. No.: US 2026/0198923 A1
(43) Pub. Date: Jul. 16, 2026

(54) VASCULAR ANASTOMOSIS SYSTEM

(71) Applicant: UNIVERSITY OF UTAH RESEARCH FOUNDATION, Salt Lake City, UT (US)

(72) Inventors: Azur AZAPAGIC, SALT LAKE CITY, UT (US); Jayant P. AGARWAL, SALT LAKE CITY, UT (US); Himanshu J. SANT, SALT LAKE CITY, UT (US); Bruce K. GALE, SALT LAKE CITY, UT (US); Jill E. SHEA, SALT LAKE CITY, UT (US); Samuel NELSON, SALT LAKE CITY, UT (US); Huizhong LI, SALT LAKE CITY, UT (US)

(21) Appl. No.: 19/128,933

(22) PCT Filed: Nov. 8, 2023
(86) PCT No.: PCT/US2023/079144

§ 371 Date: May 9, 2025

Related U.S. Application Data
(60) Provisional application No. 63/423,960, filed on Nov. 9, 2022.

(51) Int. Cl. A61B 17/11; A61B 17/00

(52) U.S. Cl. CPC A61B 17/11; A61B 2017/00486; A61B 2017/1107; A61B 2017/1121; A61B 2017/1132

(57) ABSTRACT

A vascular anastomosis system includes a first outer ring with a front side, a back side, a circumferential inner wall defining a lumen, a circumferential outer wall radially spaced from the inner wall, and an annular space between the inner wall and the outer wall. The system also includes a first inner ring configured to fit within the annular space to hold an everted end of a vessel within the annular space. The system may further comprise a second outer ring and a second inner ring, with the first and second outer rings being connectable via an interference fit. The system may also include a deployment tool configured to hold and connect the first and second ring assemblies.

### CROSS-REFERENCE TO RELATED APPLICATIONS

This application claims priority to and the benefit of U.S. Provisional Patent Application No. 63/423,960, filed Nov. 9, 2022 and titled ANASTOMOTIC COUPLING DEVICE, the entirety of which is incorporated herein by reference.

### BACKGROUND

### Technical Field

This disclosure relates to an anastomotic coupling device with force interference fit features useful for end-to-end anastomosis of arteries or veins.

### Related Technology

Microvascular anastomosis is a surgical technique that involves the connection of small blood vessels. Microvascular anastomosis is a delicate and complex procedure that requires a high degree of surgical skill and precision. Traditionally, microvascular anastomosis has been performed using hand-sutured techniques. In such techniques, the surgeon uses fine sutures to manually stitch together the ends of the vessels. While this technique has been utilized for many years, it has several limitations. For example, hand suturing is time-consuming, technically challenging, and prone to complications such as endothelial lacerations and thrombosis. Furthermore, the success of the procedure is heavily dependent on the skill and experience of the surgeon.

Other methods involve the use of microvascular anastomotic couplers. These devices are designed to mechanically join the ends of the vessels, thereby reducing the reliance on manual suturing. However, existing microvascular anastomotic couplers also have limitations. For example, they may not be suitable for all types of vessels, they can be difficult to use, and they may not consistently reduce the risk of post-surgery complications.

Accordingly, there is an ongoing need for improved and more straightforward means for vascular anastomosis with lower risk of endothelial lacerations or other risks.

### SUMMARY

The disclosed vascular anastomosis system includes a first outer ring and a first inner ring. The first outer ring has a front side, a back side, a circumferential inner wall defining a lumen, a circumferential outer wall radially spaced from the inner wall, and an annular space between the inner wall and the outer wall. The first inner ring is configured to fit within the annular space to hold an everted end of a vessel within the annular space. The first inner ring may include a non-uniform inner diameter that tapers from a larger inner diameter on a back side to a smaller inner diameter on a front side.

The system may further comprise a second outer ring and a second inner ring. The second outer ring has a front side, a back side, a circumferential inner wall defining a lumen, a circumferential outer wall radially spaced from the inner wall, and an annular space between the inner wall and the outer wall. The second inner ring is configured to fit within the annular space to hold an everted end of a second vessel within the annular space.

The first outer ring and the second outer ring may be connectable via an interference fit. The first outer ring may comprise a circumferential connecting wall connected to the outer wall of the first outer ring and extending distally therefrom. The connecting wall may be configured to at least partially encompass the outer wall of the second outer ring to form the interference fit.

The system may further comprise a deployment tool configured to hold a first ring assembly (the first outer ring and the first inner ring) and second ring assembly (the second outer ring and second inner ring). The deployment tool may be actuatable to connect the first and second ring assemblies and thereby bring the first and second vessel ends into secured contact. The deployment tool may move from an open position to a closed position by rotating the first and/or second ring assemblies through an arc to thereby bring the first and second ring assemblies into contact. The system may further comprise an anvil tool configured to assist in everting the end of a vessel into the annular space of the respective outer ring.

A method of performing a vascular anastomosis includes passing a first vessel end through a first outer ring, cutting the first vessel end longitudinally at multiple locations to create flaps, everting the first vessel end over the inner wall of the first outer ring and inserting the first vessel end into the annular space of the first outer ring, and inserting a first inner ring configured to fit within the annular space to thereby hold the everted vessel end within the annular space.

The method may further include passing a second vessel end through a second outer ring, cutting the second vessel end longitudinally at multiple locations to create flaps, everting the second vessel end over the inner wall of the second outer ring and inserting the second vessel end into the annular space of the second outer ring, inserting a second inner ring configured to fit within the annular space to thereby hold the everted vessel end within the annular space, and connecting the first and second ring assemblies via an interference fit to thereby bring the first and second vessel ends into contact. The longitudinal cuts of the first vessel end and/or the second vessel end may be spaced at about 90 degrees to form four flaps.

### BRIEF DESCRIPTION OF FIGURES

FIG. 1A is an exploded view of a portion of the vascular anastomosis system, showing a first outer ring and first inner ring.

FIG. 1B shows an end of the first vessel passed through the lumen of the first outer ring.

FIG. 1C shows flaps of the first vessel after being everted over the inner wall, toward the back side, and into the annular space.

FIG. 1D shows the first inner ring after insertion into the annular space to secure the end of the first vessel therein.

FIG. 1E shows a cross-sectional view of the first outer ring with first inner ring inserted within the annular space to secure the everted end of first vessel.

FIG. 2A shows another portion of the anastomosis system, showing the second outer ring and second inner ring.

FIG. 2B shows an end of the second vessel passed through the lumen of the second outer ring.

FIG. 2C shows a cross-sectional view of the second outer ring with second inner ring inserted within the annular space to secure the everted end of the second vessel.

FIG. 3 shows cross-sectional views of a first ring assembly formed from the first outer ring and the first inner ring, and a second ring assembly formed from the second outer ring and second inner ring.

FIGS. 4A and 4B show a deployment tool and an anvil tool that can be used to prepare and connect the first and second ring assemblies.

FIG. 5A shows a deployment device in an open position, with first holder holding the first outer ring and second holder holding the second outer ring.

FIG. 5B shows the deployment tool with ends of first and second vessels cut to form flaps.

FIG. 5C shows use of the anvil tool to assist in everting the flaps and positioning the vessel tissue in the respective annular spaces of the first outer ring and second outer ring.

FIG. 5D shows the flaps after being everted into the respective annular spaces.

FIG. 5E shows actuation of the deployment tool to bring the first outer ring and second outer ring together to be connected via interference fit to thereby bring the first vessel and the second vessel into contact.

### DETAILED DESCRIPTION

### Anastomosis System Overview

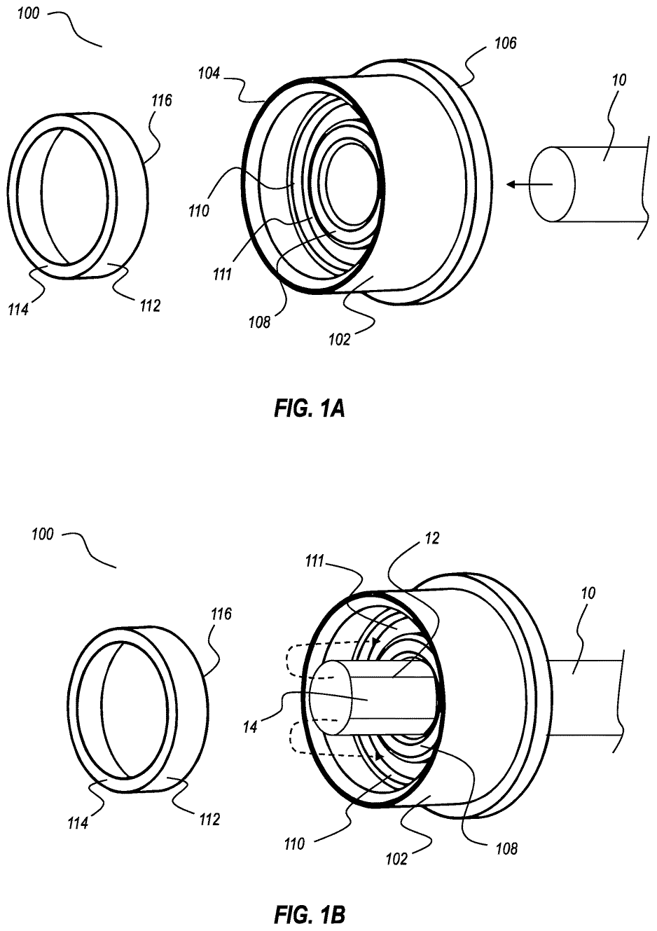

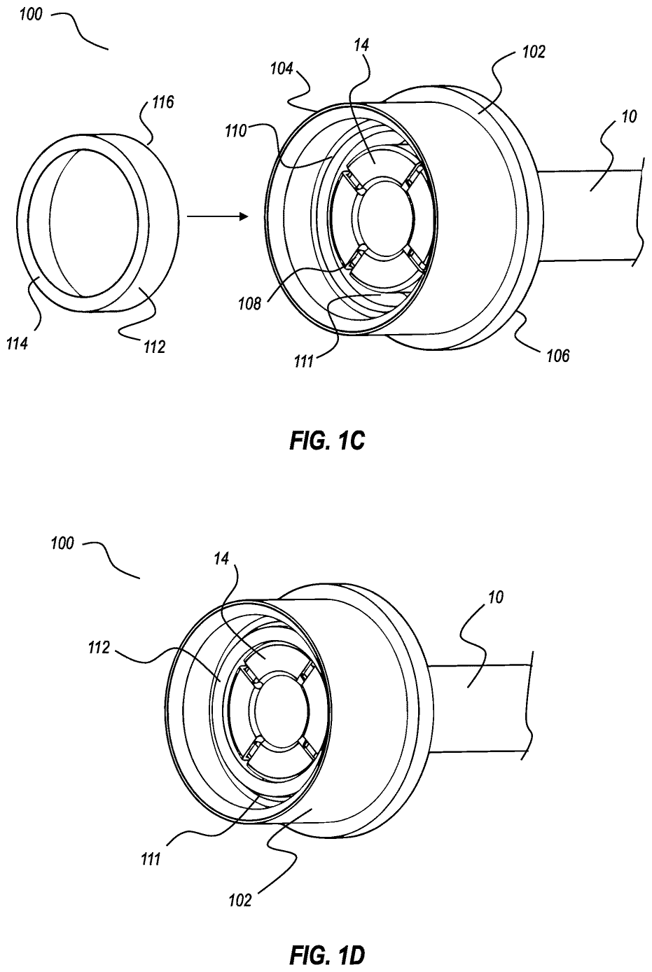

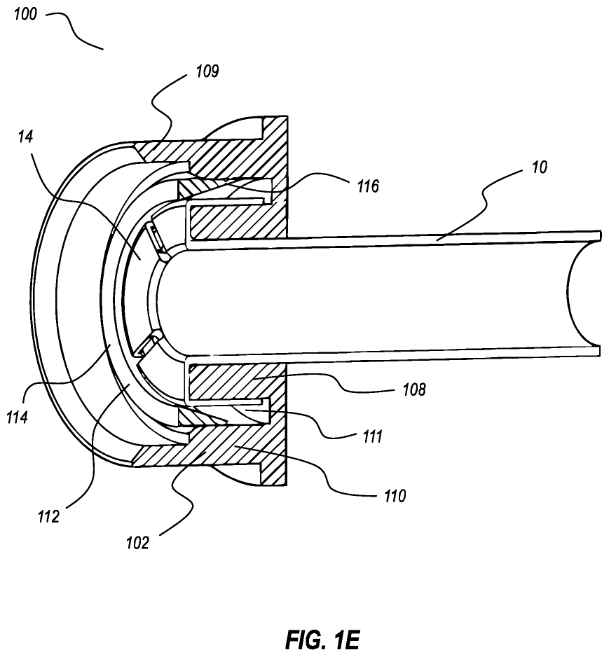

Referring to FIG. 1A, the vascular anastomosis system 100 includes a first outer ring 102 and a first inner ring 112. The first outer ring 102 has a front side 104 and a back side 106. The first outer ring 102 also includes a circumferential inner wall 108 that defines a lumen. This lumen allows a first vessel 10 to pass from the back side 106 to the front side 104. Radially spaced from the inner wall 108 is a circumferential outer wall 110. Between the inner wall 108 and the outer wall 110 is an annular space 111. This annular space 111 is configured to receive an everted end of the first vessel 10. As shown, the annular space 111 is open toward the front side 104 and closed toward the back side 106.

The first inner ring 112, which also includes a front side 114 and a back side 116 corresponding to the front side 104 and back side 106 of the first outer ring 102, is designed to fit within the annular space 111. The first inner ring 112 serves to hold the everted end of the first vessel 10 within the annular space 111, as described in more detail below. This configuration ensures a secure and reliable connection between the first vessel 10 and the first ring assembly (the first outer ring 102 and first inner ring 112) of the vascular anastomosis system 100.

In some cases, the first outer ring 102 and the first inner ring 112 may be independently made from a biocompatible material, such as a biocompatible polymer or a surgical-grade stainless steel. The specific material chosen may depend on various factors, including the specific application of the vascular anastomosis system 100, the size of the vessels to be connected, and the desired mechanical properties of the system.

The dimensions of the first outer ring 102 and the first inner ring 112, including the diameter of the lumen, the radial spacing between the inner wall 108 and the outer wall 110, and the size of the annular space 111, may vary depending on the size of the vessels to be connected. In some cases, the first outer ring 102 and the first inner ring 112 may be designed to accommodate vessels with an outside diameter ranging from 1.5 to 4.0 mm and/or a wall thickness of 0.5 mm or less.

The first inner ring 112 may be designed with a non-uniform inner diameter (e.g., tapering) that includes a larger inner diameter on the back side 116 with a smaller inner diameter on the front side 114. As explained in more detail below, this non-uniform inner diameter design may facilitate the secure holding of the everted end of the first vessel 10 when the first inner ring 112 is inserted into the annular space 111.

FIG. 1B illustrates passage of an end of the first vessel 10 through the lumen of the first outer ring 102. The first vessel 10, which can be an artery or a vein, is inserted into the lumen of the first outer ring 102 from the back side 106 towards the front side 104. The lumen is defined by the circumferential inner wall 108 of the first outer ring 102 and is sized to accommodate the outside diameter of the first vessel 10. The lumen may have a diameter that is slightly larger than the outside diameter of the first vessel 10 to facilitate the insertion of the vessel.

Once the end of the first vessel 10 is passed through the lumen, one or more longitudinal cuts 12 can be made in the first vessel 10 to generate flaps 14. These cuts 12 are made longitudinally along the length of the vessel end, and they can be spaced evenly around the circumference of the vessel. The number and spacing of the cuts 12 can be varied depending on the size and type of the vessel, the specific application of the vascular anastomosis system 100, and other factors. In some cases, the cuts 12 can be spaced at about 90 degrees to form four flaps 14, although other configurations are also possible (e.g., two cuts spaced at about 180 degrees apart, three cuts spaced at about 120 degrees apart).

After the cuts 12 are made, the flaps 14 are everted over the outer diameter of the inner wall 108 and into the annular space 111. The eversion of the flaps 14 can be facilitated by the use of an anvil tool or another suitable instrument. The everted flaps 14 are positioned within the annular space 111, which is defined between the inner wall 108 and the outer wall 110 of the first outer ring 102. The annular space 111 is sized to accommodate the everted flaps 14.

As shown in FIGS. 1C and 1D, following the eversion of the end of the first vessel 10, the first inner ring 112 is inserted into the first outer ring 102. The insertion of the first inner ring 112 into the annular space 111 serves to secure the everted end of the first vessel 10, providing a reliable and secure connection between the first vessel 10 and the vascular anastomosis system 100.

The first inner ring 112 forms an interference fit between the inner surface of the outer wall 110 of the first outer ring 102 and the everted vessel tissue lying against the outer surface of the inner wall 108. This interference fit ensures a secure and reliable connection between the first vessel 10 and the first outer ring 102, reducing the risk of disconnection or leakage. The interference fit is facilitated by the tapering design of the inner diameter of the first inner ring 112, which allows for a versatile fit within the annular space 111 for a variety of vessel thicknesses.

FIG. 1E shows a cross-sectional view of the first outer ring 102 with the first inner ring 112 inserted within the annular space 111. This configuration secures the everted end of the first vessel 10. As shown, the first inner ring 112 includes a non-uniform inner diameter that can taper from a larger inner diameter on the back side 116 to a smaller inner diameter on the front side 114.

This design facilitates the securement of vessels with a variety of vessel wall thicknesses. The user can insert the first inner ring 112 until the inner diameter engages against the everted vessel tissue. The first inner ring 112 will be inserted farther into the annular space 111 for vessels with a thinner wall thickness and will not be inserted as far into the annular space 111 for vessels with greater wall thickness. This adaptability to different vessel wall thicknesses enhances the versatility and applicability of the vascular anastomosis system 100.

On the other hand, the outer diameter of the first inner ring 112 can be substantially uniform, which simplifies the manufacturing process and ensures a consistent engagement against the inner surface of the outer wall 110.

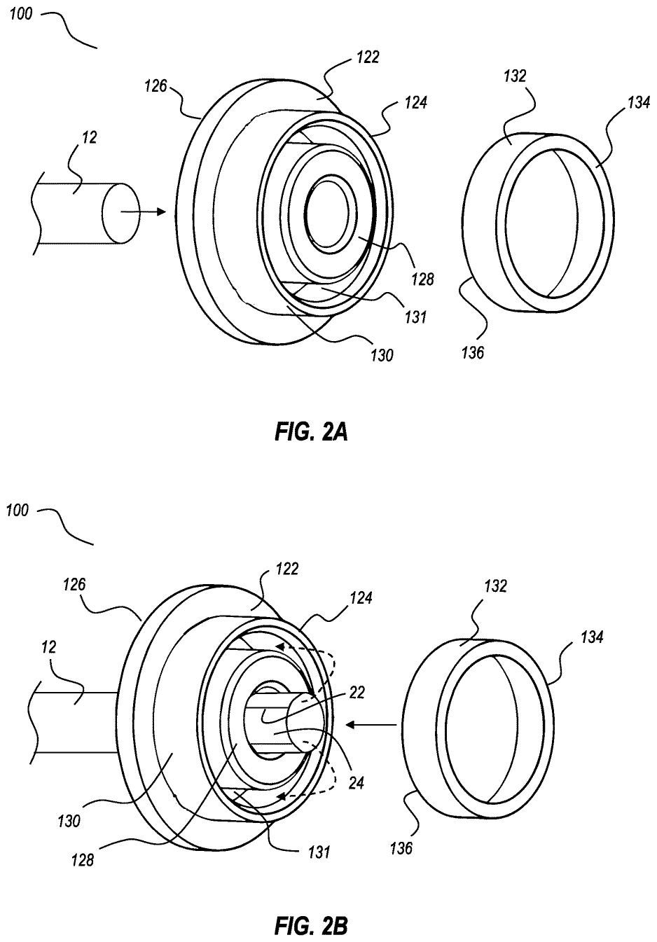

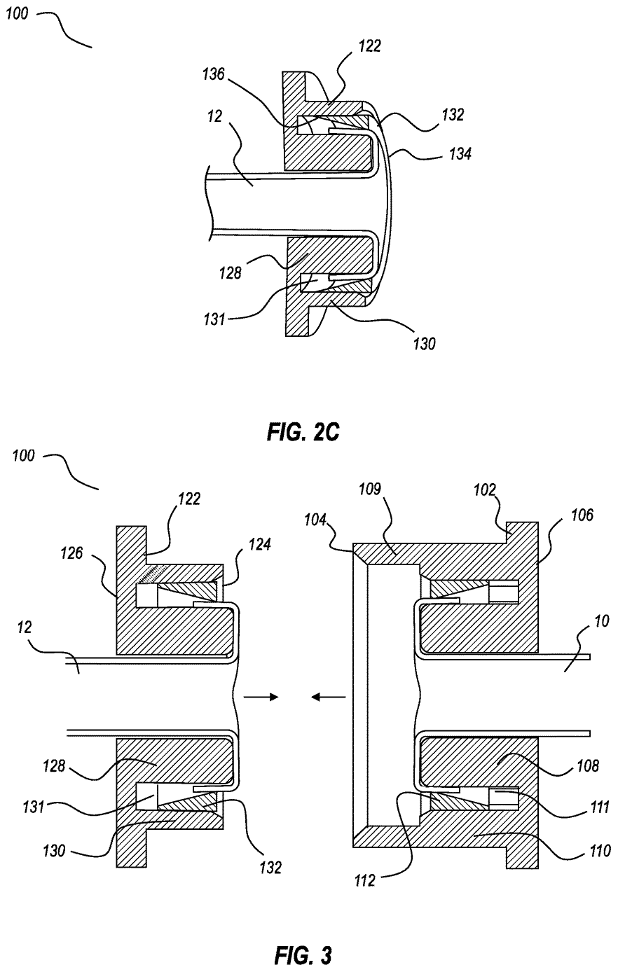

FIGS. 2A-2C illustrate the second outer ring 122 and second inner ring 132. These components include features similar to their counterparts in the first ring assembly, and the corresponding disclosure above is applicable to like components of the second ring assembly.

The second outer ring 122 has a front side 124 and a back side 126. Similar to the first outer ring 102, the second outer ring 122 includes a circumferential inner wall 128 that defines a lumen. This lumen allows a second vessel 12 to pass from the back side 126 to the front side 124. Radially spaced from the inner wall 128 is a circumferential outer wall 130. Between the inner wall 128 and the outer wall 130 is an annular space 131. This annular space 131 is configured to receive an everted end of the second vessel 12.

The second inner ring 132, which also includes a front side 134 and a back side 136 corresponding to the front side 124 and back side 126 of the second outer ring 122, is designed to fit within the annular space 131 to hold the everted end of the second vessel 12 within the annular space 131.

In some cases, the second outer ring 122 and the second inner ring 132 may be made from a biocompatible material, such as a biocompatible polymer or a surgical-grade stainless steel. The specific material chosen may depend on various factors, including the specific application of the vascular anastomosis system 100, the size of the vessels to be connected, and the desired mechanical properties of the system.

As with the first ring assembly, the dimensions of the second outer ring 122 and the second inner ring 132, including the diameter of the lumen, the radial spacing between the inner wall 128 and the outer wall 130, and the size of the annular space 131, may vary depending on the size of the vessels to be connected. The second outer ring 122 and the second inner ring 132 may be designed to accommodate vessels with an outside diameter ranging from 1.5 to 4.0 mm and a wall thickness of 0.5 mm or less.

As with the first inner ring 112, the second inner ring 132 may be designed with a non-uniform inner diameter that tapers from a larger inner diameter on the back side 136 to a smaller inner diameter on the front side 134. The first inner ring 112 and second inner ring 132 may be identical.

FIG. 2B shows passage of an end of the second vessel 12 through the lumen of the second outer ring 122 from the back side 126 towards the front side 124. Once the end of the second vessel 12 is passed through the lumen, one or more longitudinal cuts 22 are made in the second vessel 12 to generate flaps 24. The flaps 24 are everted over the outer diameter of the inner wall 128 and into the annular space 131. Eversion of the flaps 24 can be facilitated by the use of an anvil tool or another suitable instrument.

FIG. 2C illustrates, in cross-section, the second inner ring 132 after insertion into the second outer ring 122. As with the first ring assembly, the second inner ring 132 is designed to fit within the annular space 131 of the second outer ring 122 and form an interference fit between the inner surface of the outer wall 130 of the second outer ring 122 and the everted vessel tissue lying against the outer surface of the inner wall 128. The interference fit is also facilitated by the non-uniform inner diameter of the second inner ring 132 (e.g., tapered from a larger inner diameter on the back side 136 to a smaller inner diameter on the front side 134), which enables a secure fit against a variety of vessel thicknesses by varying the depth at which the inner ring 132 is inserted into the annular space 131.

FIG. 3 depicts a cross-sectional view of the first and second ring assemblies following securement of the opposing vessel ends. The first outer ring 102 and the second outer ring 122 are designed to be connectable via an interference fit, which ensures a secure and reliable connection between the two ring assemblies.

The first outer ring 102 comprises a circumferential connecting wall 109 that is connected to the outer wall 110 of the first outer ring 102 and extends distally (i.e., toward the front side 104) therefrom. The connecting wall 109 is configured to at least partially encompass the outer wall 130 of the second outer ring 122 when these components form a face-to-face connection.

This design allows the first outer ring 102 and the second outer ring 122 to be securely connected via an interference fit. The inner diameter of the connecting wall 109 can be substantially similar to the outer diameter of the outer wall 130 of the second outer ring 122. This design enables a tight fit between the connecting wall 109 of the first outer ring 102 and the outer wall 130 of the second outer ring 122, thereby enhancing the security and reliability of the connection between the first and second ring assemblies.

The design also beneficially enables connection of the first outer ring 102 and the second outer ring 122 without requiring a specific radial alignment of these components. That is, the first outer ring 102 can be rotated relative to the second outer ring 122, and vice versa, without impacting the ability to connect the two components, so long as they face each other and are connected to form the interference fit. This advantageously avoids extra steps involving rotating and aligning separate pieces for proper connection, as is the case with many prior anastomotic devices that utilize connecting arms and corresponding grooves, for example.

Deployment Tool & Anvil Tool

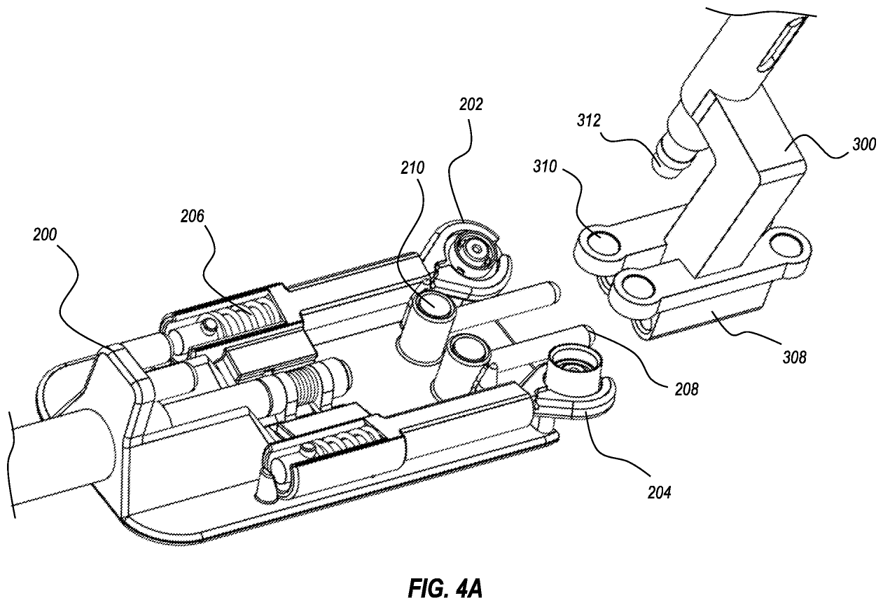

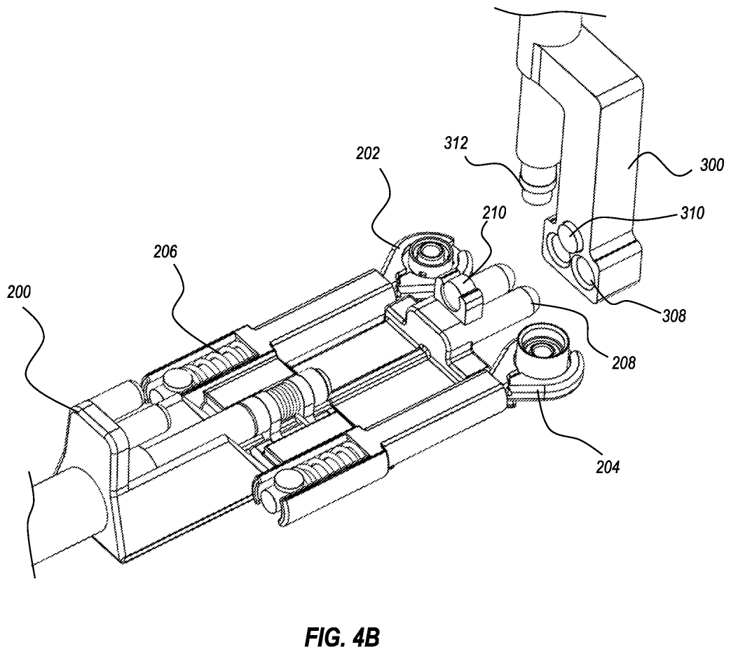

Referring to FIG. 4A, the vascular anastomosis system 100 can include a deployment tool 200 and an anvil tool 300 that can assist in connecting the first and second ring assemblies. The illustrated deployment tool 200 includes a first holder 202 and a second holder 204, which are configured to securely hold the first and second ring assemblies, respectively. The deployment tool 200 is actuatable to connect the first and second ring assemblies and thereby bring the first and second vessel ends into secured contact.

The illustrated deployment tool 200 includes a biasing element 206, such as one or more torsion springs (as shown), leaf springs, and/or other springs. The biasing element 206 serves to bias the device toward an open or closed position. The deployment tool 200 can move from the open position to the closed position by rotating the first and/or second ring assemblies through an arcuate path. This rotation brings the first and second ring assemblies into contact, thereby facilitating the connection of the first and second vessel ends. The arcuate closing motion allows the first and second ring assemblies to lie relatively flat when in the open position, which provides easier vessel manipulation and easier access of the anvil tool 300 to the ring assemblies, and then to close in a position that positions the joined vessel appropriately.

Actuation of the deployment tool 200 can additionally or alternatively be achieved through various mechanisms, such as a mechanical linkage, a hydraulic system, an electric motor, magnetic couplings, or other suitable actuation mechanism. The actuation mechanism is designed to provide a controlled movement of the first and second ring assemblies, ensuring a secure and reliable connection between the first and second vessel ends. The actuation mechanism can be designed to provide a range of motion that is sufficient to bring the first and second ring assemblies into contact, but not so large as to cause damage to the vessels or the vascular anastomosis system 100.

The deployment tool 200 also includes a connector 208 for receiving the anvil tool 300. The connector 208 serves to align the anvil tool 300 with respect to the first and/or second ring assemblies. This alignment enables the actuation of the anvil tool 300 at the proper position. The connector 208 can engage with a corresponding connector 308 of the anvil tool 300. The connectors 208 and 308 can include a rail and slot system (as shown), a screw mechanism, linear bearings, telescopic slides, belt and pulley system, and/or other linear guide system.

The anvil tool 300 is another mechanical device used in the vascular anastomosis system 100. When actuated, the anvil tool 300 presses an anvil 312 against the vessel end. This action can press the vessel tissue into the annular space of the respective outer ring, thereby facilitating the eversion of the vessel end and its securement within the annular space.

The anvil tool 300 can be detached from the deployment tool 200, rotated, and re-inserted onto the deployment tool 200. This feature allows the position of the anvil 312 to be switched with respect to the first and second ring assemblies, providing flexibility and adaptability in the operation of the vascular anastomosis system 100.

The deployment tool 200 includes a lock element 210 for engaging with a corresponding lock element 310 of the anvil tool 300 when properly positioned. This locking mechanism ensures a secure connection between the deployment tool 200 and the anvil tool 300, preventing unintentional disconnection during operation of the anvil tool 300. The lock elements 210, 310 can include magnets for magnetic coupling, clamps, mating friction fit elements, threaded connections, clips, suction cups, hook and loop fastener materials, and/or other suitable fastening devices. In the illustrated embodiment, the lock elements 210, 310 comprise magnets for forming magnetic couplings when the anvil tool 310 is positioned on the deployment tool 200.

FIG. 4B illustrates another configuration of the deployment tool 200 and anvil tool 300. The configuration shown in FIG. 4B is similar to the configuration of FIG. 4A, and the above description applies to FIG. 4B as well. In FIG. 4B, the lock elements 210, 310 are oriented so as to couple in a horizontal position rather than the vertically stacked position of the FIG. 4A configuration.

### Example Anastomosis Method

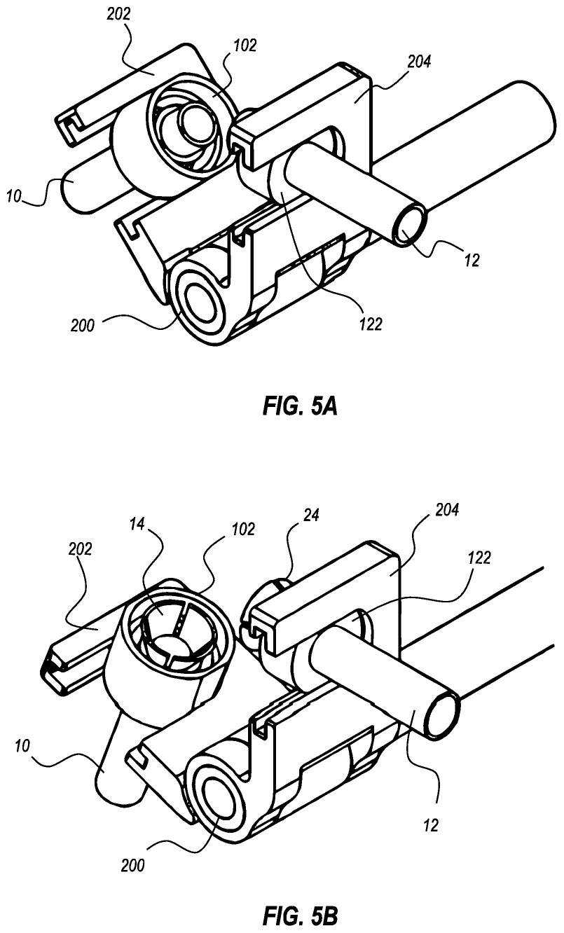

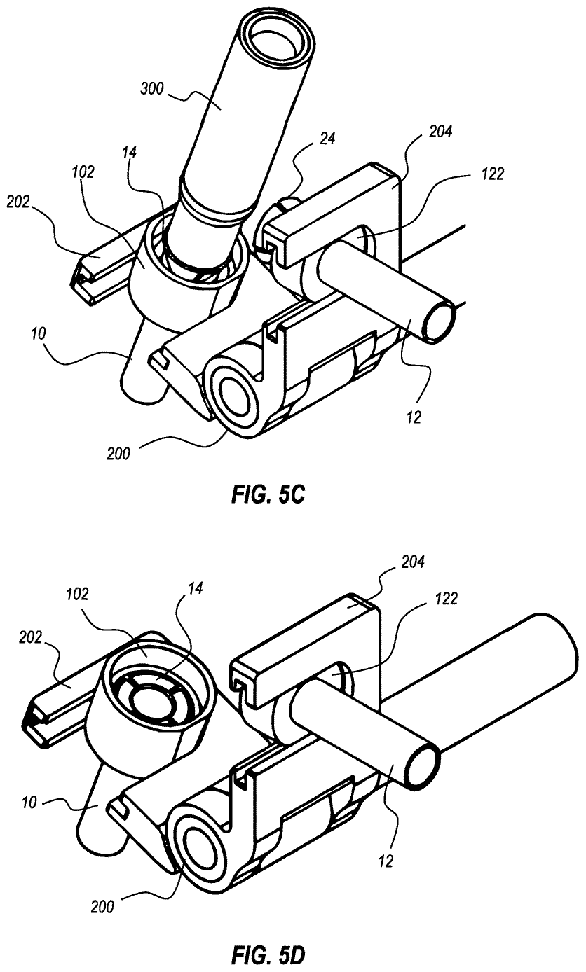

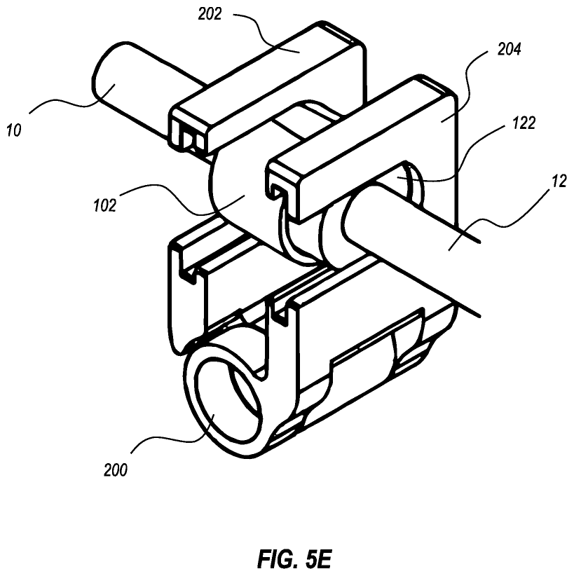

FIGS. 5A-5E illustrate a deployment device 200 with a slightly different configuration, but which incorporates many of the same principles discussed above.

Referring to FIG. 5A, the deployment device 200 is shown with the first holder 202 holding the first outer ring 102 and the second holder 204 holding the second outer ring 122. The first holder 202 and the second holder 204 of the deployment device 200 are designed to securely hold the first outer ring 102 and the second outer ring 122, respectively. These holders can be designed with a specific shape and size to accommodate the corresponding outer rings.

Referring to FIG. 5B, the deployment tool 200 is shown with the end of the first vessel 10 cut to form flaps 14 and the end of the second vessel 12 cut to form flaps 24.

Referring to FIG. 5C, the anvil tool 300 is used to assist in everting the flaps 14 and 24 to position the vessel tissue in the respective annular spaces of the first outer ring 102 and the second outer ring 122. The anvil tool 300 can be actuated to press the anvil against the vessel end and press the vessel tissue into the annular space of the respective outer ring, thereby facilitating the eversion of the vessel end and its securement within the annular space.

Referring to FIG. 5D, the flaps 14 are shown after being everted into the annular space of the first outer ring 102. Flaps 24 have also been everted into the annular space of the second outer ring 122, though the flaps 24 are not visible in this view.

In some cases, the size and shape of the flaps 14, 24 can be varied depending on the size and type of the vessel, the specific application of the vascular anastomosis system 100, and other factors. For instance, the flaps 14, 24 can be made larger or smaller, or they can be shaped differently, to accommodate different sizes and types of vessels. The flaps 14, 24 can also be everted to different extents, depending on the desired fit within the annular space.

Referring to FIG. 5E, the deployment tool 200 is actuated to bring the first outer ring 102 and the second outer ring 122 together. Upon actuation, the first outer ring 102 and the second outer ring 122 are brought together to be connected via an interference fit. The interference fit ensures a secure and reliable connection between the first and second ring assemblies, reducing the risk of disconnection or leakage.

### Example Embodiments

The following clauses represent a non-exhaustive list of example embodiments.

Clause 1. An anastomosis system for coupling ends of a vessel, the system comprising: a first outer ring comprising a front side and a back side, a circumferential inner wall defining a lumen through which a vessel end is passable from the back side to the front side, a circumferential outer wall radially spaced from the inner wall, and an annular space between the inner wall and the outer wall in which the vessel end can be everted; and a first inner ring configured in size and shape to fit within the annular space to thereby hold the everted vessel end within the annular space.

Clause 2. The system of clause 1, wherein an inner diameter of the outer wall of the first outer ring and an outer diameter of the first inner ring form a friction fit.

Clause 3. The system of clause 1 or claim 2, wherein the annular space opens to the front side and is closed to the back side.

Clause 4. The system of any one of clauses 1-3, wherein the first inner ring has a non-uniform inner diameter.

Clause 5. The system of clause 4, wherein the inner diameter of the first inner ring tapers.

Clause 6. The system of clause 5, wherein the inner diameter of the first inner ring tapers from a larger inner diameter on a back side to a smaller inner diameter on a front side, the front side and back side of the first inner ring corresponding to the front side and back side of the first outer ring.

Clause 7. The system of any one of clauses 1-6, further comprising: a second outer ring, the second outer ring comprising a front side and a back side, a circumferential inner wall defining a lumen through which a vessel end is passable from the back side to the front side, a circumferential outer wall radially spaced from the inner wall, and an annular space between the inner wall and the outer wall in which the vessel end can be everted; and a second inner ring configured in size and shape to fit within the annular space to thereby hold the everted vessel end within the annular space, wherein the first and second outer rings are connectable.

Clause 8. The system of clause 7, wherein the first and second outer rings are connectable via friction fit.

Clause 9. The system of clause 7 or claim 8, wherein the first outer ring comprises a circumferential connecting wall connected to the outer wall and extending distally therefrom, the connecting wall being configured to at least partially encompass the outer wall of the second outer ring.

Clause 10. The system of clause 9, wherein an inner diameter of the connecting wall is substantially similar to an outer diameter of the outer wall of the second outer ring.

Clause 11. The system of any one of clauses 7-10, wherein the first and second inner rings are identical.

Clause 12. The system of any one of clauses 7-11, wherein the first and second outer rings are connectable by joining respective front sides to thereby bring separate vessel ends into contact.

Clause 13. The system of any one of clauses 7-12, wherein the first outer ring and second outer ring are configured to connect without requiring specific radial alignment.

Clause 14. The system of any one of clauses 7-13, wherein the first outer ring and first inner ring form a first ring assembly for holding a first vessel end, and wherein the second outer ring and second inner ring form a second ring assembly for holding a second vessel end, wherein the system further comprises a deployment tool configured to hold the first and second ring assemblies, the deployment tool being actuatable to connect the first and second ring assemblies and thereby bring the first and second vessel ends into secured contact, wherein the deployment tool is adjustable between an open position wherein the first and second ring assemblies are separated and a closed position wherein the first and second ring assemblies are brought into contact and connected.

Clause 15. The system of clause 14, wherein the deployment tool moves from the open position to the closed position by rotating the first and/or second ring assemblies through an arc to thereby bring the first and second ring assemblies into contact.

Clause 16. The system of clause 14 or clause 15, further comprising an anvil tool configured to insert a vessel end into the annular space of the respective first outer ring or second outer ring.

Clause 17. The system of clause 16, wherein the deployment tool includes a connector for receiving the anvil tool and aligning the anvil tool with respect to the first and/or second ring assemblies to enable actuation of the anvil tool to insert the vessel end into the annular space of the respective outer ring.

Clause 18. A method of performing a vascular anastomosis, optionally using a system as in any one of clauses 1-17, the method comprising: passing a first vessel end through a first outer ring, the first outer ring comprising a circumferential inner wall defining a lumen through which the first vessel end is passed, a circumferential outer wall radially spaced from the inner wall, and an annular space between the inner wall and the outer wall; cutting the first vessel end longitudinally at multiple locations to create flaps; everting the first vessel end over the inner wall of the first outer ring and inserting the first vessel end into the annular space of the first outer ring; and inserting a first inner ring configured in size and shape to fit within the annular space to thereby hold the everted vessel end within the annular space, the first outer ring and first inner ring forming a first ring assembly.

Clause 19. The method of clause 18, further comprising: passing a second vessel end through a second outer ring, the second outer ring comprising a circumferential inner wall defining a lumen through which the second vessel end is passed, a circumferential outer wall radially spaced from the inner wall, and an annular space between the inner wall and the outer wall; cutting the second vessel end longitudinally at multiple locations to create flaps; everting the second vessel end over the inner wall of the second outer ring and inserting the second vessel end into the annular space of the second outer ring; inserting a second inner ring configured in size and shape to fit within the annular space to thereby hold the everted vessel end within the annular space, the second outer ring and second inner ring forming a second ring assembly; and connecting the first and second ring assemblies via a friction fit to thereby bring the first and second vessel ends into contact.

Clause 20. The method of clause 19, wherein longitudinal cuts of the first vessel end and/or the second vessel end are spaced at about 90 degrees to form four flaps.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An anastomosis system for coupling ends of a vessel, the system comprising:
a first outer ring comprising
a front side and a back side,
a circumferential inner wall defining a lumen through which a vessel end is passable from the back side to the front side,
a circumferential outer wall radially spaced from the inner wall, and
an annular space between the inner wall and the outer wall in which the vessel end can be everted; and
a first inner ring configured in size and shape to fit within the annular space to thereby hold the everted vessel end within the annular space.

2. The system of claim 1, wherein an inner diameter of the outer wall of the first outer ring and an outer diameter of the first inner ring form a friction fit.

3. The system of claim 1, wherein the annular space opens to the front side and is closed to the back side.

4. The system of claim 1, wherein the first inner ring has a non-uniform inner diameter.

5. The system of claim 4, wherein the inner diameter of the first inner ring tapers.

6. The system of claim 5, wherein the inner diameter of the first inner ring tapers from a larger inner diameter on a back side to a smaller inner diameter on a front side, the front side and back side of the first inner ring corresponding to the front side and back side of the first outer ring.

7. The system of claim 1, further comprising:
a second outer ring, the second outer ring comprising
a front side and a back side,
a circumferential inner wall defining a lumen through which a vessel end is passable from the back side to the front side,
a circumferential outer wall radially spaced from the inner wall, and
an annular space between the inner wall and the outer wall in which the vessel end can be everted; and
a second inner ring configured in size and shape to fit within the annular space to thereby hold the everted vessel end within the annular space,
wherein the first and second outer rings are connectable.

8. The system of claim 7, wherein the first and second outer rings are connectable via friction fit.

9. The system of claim 7, wherein the first outer ring comprises a circumferential connecting wall connected to the outer wall and extending distally therefrom, the connecting wall being configured to at least partially encompass the outer wall of the second outer ring.

10. The system of claim 9, wherein an inner diameter of the connecting wall is substantially similar to an outer diameter of the outer wall of the second outer ring.

11. The system of claim 7, wherein the first and second inner rings are identical.

12. The system of claim 7, wherein the first and second outer rings are connectable by joining respective front sides to thereby bring separate vessel ends into contact.

13. The system of claim 7, wherein the first outer ring and second outer ring are configured to connect without requiring specific radial alignment.

14. The system of claim 7, wherein the first outer ring and first inner ring form a first ring assembly for holding a first vessel end, and wherein the second outer ring and second inner ring form a second ring assembly for holding a second vessel end, wherein the system further comprises a deployment tool configured to hold the first and second ring assemblies, the deployment tool being actuatable to connect the first and second ring assemblies and thereby bring the first and second vessel ends into secured contact, wherein the deployment tool is adjustable between an open position wherein the first and second ring assemblies are separated and a closed position wherein the first and second ring assemblies are brought into contact and connected.

15. The system of claim 14, wherein the deployment tool moves from the open position to the closed position by rotating the first and/or second ring assemblies through an arc to thereby bring the first and second ring assemblies into contact.

16. The system of claim 14, further comprising an anvil tool configured to insert a vessel end into the annular space of the respective first outer ring or second outer ring.

17. The system of claim 16, wherein the deployment tool includes a connector for receiving the anvil tool and aligning the anvil tool with respect to the first and/or second ring assemblies to enable actuation of the anvil tool to insert the vessel end into the annular space of the respective outer ring.

18. A method of performing a vascular anastomosis, optionally using a system as in claim 1, the method comprising:
passing a first vessel end through a first outer ring, the first outer ring comprising a circumferential inner wall defining a lumen through which the first vessel end is passed, a circumferential outer wall radially spaced from the inner wall, and an annular space between the inner wall and the outer wall;
cutting the first vessel end longitudinally at multiple locations to create flaps;
everting the first vessel end over the inner wall of the first outer ring and inserting the first vessel end into the annular space of the first outer ring; and
inserting a first inner ring configured in size and shape to fit within the annular space to thereby hold the everted vessel end within the annular space, the first outer ring and first inner ring forming a first ring assembly.

19. The method of claim 18, further comprising:
passing a second vessel end through a second outer ring, the second outer ring comprising a circumferential inner wall defining a lumen through which the second vessel end is passed, a circumferential outer wall radially spaced from the inner wall, and an annular space between the inner wall and the outer wall;
cutting the second vessel end longitudinally at multiple locations to create flaps;
everting the second vessel end over the inner wall of the second outer ring and inserting the second vessel end into the annular space of the second outer ring;
inserting a second inner ring configured in size and shape to fit within the annular space to thereby hold the everted vessel end within the annular space, the second outer ring and second inner ring forming a second ring assembly; and
connecting the first and second ring assemblies via a friction fit to thereby bring the first and second vessel ends into contact.

20. The method of claim 19, wherein longitudinal cuts of the first vessel end and/or the second vessel end are spaced at about 90 degrees to form four flaps.
